(19) United States
(12) Patent Application Publication
(10) Pub. No.: US 2012/0006334 A1
(43) Pub. Date: Jan. 12, 2012

(54) ROTATING SEX MACHINE

(76) Inventors: Nelson Cayabyab, Long Beach, CA (US); Ernesto Cayabyab, Los Angeles, CA (US)

(21) Appl. No.: 12/833,806

(22) Filed: Jul. 9, 2010

(51) Int. Cl. A61G 15/00; A61F 5/00

(52) U.S. Cl. 128/845; 600/38

(57) ABSTRACT

An apparatus for use in performing intercourse, including: a horizontal support designed to support a female in the supine position; a bearing assembly attached to the outside of the horizontal support; and a base horizontally slidably attached to the bottom of the bearing assembly. The bearing assembly is designed to permit rotation of the horizontal support about the axis of rotation of the bearing assembly. The horizontal support and bearing assembly are designed so that this axis of rotation is coaxial with the longitudinal axis of the sex organ of the female when she is supported on the horizontal support in the supine position. The heights of the horizontal support and base are adjustable. Thus the height of the female's sex organ can be adjusted for penetration by a male partner's sex organ when the male partner is standing at the correct end of the horizontal support.

### BACKGROUND OF THE INVENTION

(1) Field of the Invention

The present invention relates to the field of sexual aids and more particularly to machines that provide means for rotary stimulation during sex.

(2) Description of the Related Art

Sex is one of mankind's ultimate forms of natural pleasure and is also crucial for mankind's survival. In fact, if we Google the words ultimate pleasure, we will see that all references will be about sex and intimacy. Sex is universally practiced by every capable individual regardless of culture and race. Various studies emphasize how important sex is in a relationship. Sex is also considered to be the ultimate form of pleasure that a man can ever experience naturally.

Sexual intimacy is the single moment in which a man and woman experience various chemical and biological processes that affects their bodies in a very positive way. Perhaps this is the reason why, according to a report, the most expensive purchases made by men are ultimately linked to sex. Some of these indirect purchases are expensive cars, jewelry and even paying for frame to make it easy for sex to happen either from casual acquaintance or from a spouse.

In one study, in a recently published book, it was even mentioned that sex is one of the most important parts of a relationship. In this same book, (promoted in CBS' The Saturday Early Show) the author explained why married couples should have sex and how to do it better. This study together with many other studies done by acknowledged authorities in Marital Relationships, Stress Relief, Personality Development and more, concur that good sex enhances a relationship and improves a person's mood, both short term and long term. Consequently, each of these studies are united in saying that exploring new ways of performing sex is among the best way, not only to enjoy it but also to sustain the enthusiasm for it.

Since the beginning of time, numerous coupling positions have been introduced by men of various cultures and races. From the most familiar position, called the missionary position, to other positions that even imitate animals. While there are other ways to enjoy sex, the only way that can result into the impregnation of a woman naturally is by the penetration of the male organ into the female organ and the release of the sperm inside the female organ. A superior sperm will then fertilize the egg which in turn will ultimately result into the creation of another human being. This method of penetration by the male organ of the female organ and then the repeated in and out action sums up all the position mankind has known in the sex act. We may read or discover a new method of performing sex but it will almost surely involve the same act in which the male organ will go in and out of the female organ. This act has remained the same since the beginning of time to now.

Not surprisingly the field of sexual aids has been a fertile one for inventors. U.S. Pat. No. 5,294,176 is directed to a sexual device for handicapped men with limited mobility of the lower part of the trunk. The device is intended to put the male partner of a sexual union more largely in control of the act and thus allows him to feel more independent with great psychological benefits. Referring to the Figures, the device includes a support member 10 with a seating platform 12 wherein the support member 10 comprises a pair of vertical legs 14, the legs being adjustable in height so as to accommodate a variety of individuals. The seating platform may include a backrest 30, a footrest 32, and also may include handles 34 which facilitate the manipulation of the device by the male member during use. The seating platform 12 also includes an annular member 28 which may be lowered and engaged with balls or casters when supported by support 10 so as to be rotatable with respect to the support 10. To use the device, the male partner lies supine on a surface such as a bed or floor and places the device across the lower portion of his trunk centered roughly above his genital area. The female partner is then seated on platform 12 utilizing backrest 30 and footrest 32 positioning herself so that the male partner may insert his sexual organ into that of his female partner and achieve sexual intimacy. The device then allows for two different kinds of motion to maximize the satisfaction of both partners. The motion may be created by the man's arms or a motorized or mechanical device may also be used. The two types of motion possible are a rotational motion in the horizontal plane which comes about when the seating platform rotates about a vertical axis at any desired angular rate and frequency. The second type of available motion is the rocking back and forth of the base about the base of legs 40. As already stated, the legs 40 are of a height and may be adjusted to insure proper penetration of the female sex organ by the male sex organ during intercourse.

U.S. Pat. No. 5,971,480 is directed to spinning and/or swinging, hanging seat for erotic purposes. The seat element is suspended from two parallel poles which are in turn suspended from the ends of a third pole with the third pole being suspended by means of the ropes from a fixed point. In the first embodiment, one participant utilizes the seat system with the man lying on his back beneath the seat while the woman floats cross-legged above him. The hole 14 in the seat 13 makes it possible for the male and female partners' genital systems to access one another to achieve intimacy. Since the seat system is suspended at a single point with a spring 21 in the system, it is possible to achieve various motions, such as spinning, swinging, bobbing, and shaking. For example, when the woman is rotated, the rope is wound and when released, the woman is rotated in the reverse direction.

U.S. Pat. No. 4,825,855 is directed to an intercourse aiding apparatus utilizing a rigid frame supported above a bed and suspended by cables. The suspended cloth panel has an opening 32 that exposes the genitalia of a user seated thereupon in a spaced relationship above the bed where the second partner is positioned. In use, the woman sits in seat 44 with her genitalia extending downwardly and exposed through opening 32 for contact with her male partner lying beneath her on bed 26. The female partner's feet may rest on bed 26 so that she may rock herself or otherwise move according to her and her partner's sexual taste. It is obvious from the description given that both reciprocating and rotational motions may be achieved between the two partners relative to one another. U.S. Pat. Nos. 7,018,329 and 5,782,243 also describe devices that make it possible to achieve both rotational and reciprocating motions between male and female partners engaging in sexual intercourse.

US Patent Application Publication No. 2003/0221692 is directed to a sexual aid turntable system which may be used for providing a new and unusual platform for engaging in sexual acts. Referring to the Figures, the device comprises a turntable system 10 with a platform 20 and an aperture 22 at the center of platform 20. The platform 20 is of sufficient thickness to bear the weight of an individual using the device and the platform 20 is attached to a ball thrust bearing assembly 30 attached to a supporting frame 20 which includes adjustable feet 50. The adjustable feet 50 may be used to adjust the height of the platform to a height that may be comfortable to the users of the device. In use, a male participant may lie on his back with the aperture 22 of platform 20 positioned above the genital area of the male user. A second participant may then sit on the platform 20 in a manner such as the genital area of the second participant is located above the aperture area 22 so that the two participants may then participate in mutual sexual activity. During this activity, the platform may then be manually rotated to make it possible for one participant to rotate about the second participant in a 360° rotational manner.

U.S. Pat. No. 5,875,779 is directed to an arcuately reciprocating human sexual fitness machine. The device comprises a pair of seats, one for the male and one for the female, and provides for the female occupant to pivot and reciprocate along an arcuate path forwardly about a horizontal axis and away from the other seat and may also reciprocate along a horizontal path. Referring to the Figures, the female figure F is seated in female seat 12 and the male figure M is seated in the male seat 14 while both seats are supported for longitudinal and rectilinear movement along rail 20 as shown in FIGS. 6 and 7. The housing 26 and the rollers 30 together with dolly assembly 40 for female seat 12 is identical in structure and function to the rolling support dolly 40 for the male seat 14. Positioned within the stanchion 42 is rotatable solid pedestal 46 which is resiliently urged within the stanchion 42 by means of coiled spring at the lower end of the pedestal. Thus, the seats have both rotational movement about a vertical axis and have a limited vertical movement as well. The seated female sits comfortably on seat 12 with her legs positioned to straddle the seated male and allowing the female partner to tilt arcuately forwardly from an upright position as seen in FIG. 2 and to tilt or pivot about a horizontal axis 74 from the horizontal plane to a degree of about 30°. The male seat is connected to a flexible extensible cord 97 and attached to the movable male seat dolly 40 in order to permit the male to vary his longitudinal position with only the requirement of pushing rearward with his feet or hands urging the movement of the dolly. The ability for the female to move through an arc while simultaneously achieving longitudinal movement along with optional up and down motion that is permitted by pedestal 46 enables the female to have greater or lesser penetration of the male sexual organ into her own sexual organ and at the same time, she may move horizontally with the assistance of dolly 40 beneath her seat. Since the male partner is able to experience similar motions, the two are able to join together in a joint rotating action, both counterbalanced as they move together in a coordinated stroking action. These actions do not have to be performed simultaneously, although it is possible to do so. Thus, the machine is capable of allowing for enormous controlled movement of a couple engaged in the marital act. U.S. Pat. No. 5,385,154 to the same inventors is also included and provides for a two seat assembly that allows for a variety of motions, essentially in a horizontal plane, while the couple is engaged in sexual intercourse.

U.S. Pat. No. 6,902,525 describes a stimulation device that provides both rotary and reciprocating motion, but is not intended for use by couples mutually engaged in the sexual act.

The invention of this device will finally allow both man and woman to experience a new method of having sex in an efficient and comfortable way. Thus, it is the hope of the inventors, that with the introduction of this new device, every couple will have a renewed interest in intimacy resulting in an improved relationship. It is also the inventor's hope to restore the fun and excitement derived from sex to couples who have been married for many years.

Before we ask what kind of a device this is, we must first ask, what new method this device promises the couple to achieve. As mentioned earlier, the only known method of intercourse mankind has performed consistently and comfortably is the act of the male organ going in and out of the female organ. Whatever position we have come to know or even practice still goes back to this basic act of reciprocating motion of the male organ inside the female organ. A totally different method that has never been used involves rotation of the female organ around the male organ once it has penetrated the female organ thus providing rotating friction instead of reciprocating friction.

An article of a very popular sex therapist concluded that the most erotic, exciting and stimulating sex position is one wherein the male partner will turn around in circles while his organ is inside the female partner. This act is different in that instead of the female partner rotating, it is the male partner who is turning around in circles. This imitates the effect of a very popular dildo that spins like a drill as it vibrates. The only problem with this position is that due to its awkwardness, it is very difficult for the male and female to perform this act continuously and even repeatedly. While this may arguably be the most difficult position without the aid of a device, the experience can be very rewarding according to the popular sex therapist.

What is needed is a device that will allow the couple to perform a rotational sex, although in a reversed role, efficiently and comfortably even if the couple is not athletic. This will be revolutionary for mankind. This is because the couple will experience a totally different sensation, one that will permit the male organ to explore the female organ in a totally different way. Since the pattern of movement is now different from what we have been used to, the experience will now be different as well. The inside of the female organ will now be explored in a way that has never been done before. The male organ will now be able to explore in a variety of ways, as the device will now permit the male organ to move in various directions thus experiencing new paths and new levels of friction. For the female partner, a testament to the ultimate pleasure it brings is the popularity of the vibrator that imitates a spinning male organ. Instead of simply moving in and out, this vibrator rotates continuously like a drill thus providing a fully stimulating sensation to the female user.

Thus, with this new invention, the female will now experience the same sensation, but this time, not from a drill-like vibrator but from her own partner. With the aid of this invention, man and woman will now be able to perform such method of intercourse comfortably, efficiently and most of all, in a very enjoyable and exciting fashion. This is because the device will not only allow the couple to perform a very stimulating new method of intercourse but it will provide the same fun, excitement and thrill that we derive only from the thrill rides experienced in many of those amusement parks we've been to. This is because the invention requires the female partner to ride inside the device and the movement of the device will be controlled both by the male and female partner for mutual enjoyment.

Development of a device which will permit rotational sex represents a great improvement in the field of sexual aids and satisfies a long felt need of couples desiring a new way to experience intimacy.

### SUMMARY OF THE INVENTION

The present invention is an apparatus for use in performing intercourse. It includes: a horizontal support designed to support a female in the supine position; a bearing assembly attached to the outside of the horizontal support; and a base horizontally slidably attached to the bottom of the bearing assembly. The bearing assembly is designed to permit rotation of the horizontal support about the axis of rotation of the bearing assembly. The horizontal support and bearing assembly are designed so that this axis of rotation is coaxial with the longitudinal axis of the sex organ of the female when she is supported on the horizontal support in the supine position. The heights of the horizontal support and base are adjustable. Thus the height of the female's sex organ can be adjusted for penetration by a male partner's sex organ when the male partner is standing at the correct end of the horizontal support.

This invention is also a method of performing intercourse comprising the steps of:

providing a horizontal support designed to support a female in the supine position;

providing a bearing assembly designed to permit rotation of the horizontal support about the axis of rotation of the bearing assembly;

attaching the bearing assembly to the outside of the horizontal support;

providing a base; and slidably attaching the base to the bottom of the bearing assembly.

The horizontal support and bearing assembly are designed so that the axis of rotation is coaxial with the longitudinal axis of the sex organ of the female when she is supported on the horizontal support in the supine position. The heights of the base and the horizontal support are adjustable. Thus the height of the female's sex organ can be adjusted for penetration by a male partner's sex organ when the male partner is standing at the proper end of the horizontal support.

Thus, during intercourse, the female partner can be rotated, moved backwards and forwards, or rotated and moved backwards and forwards (twirled).

Preferably, the horizontal support includes one or more cushions, which may be removable. Preferably the bearing assembly is also openable. The apparatus may further include a video camera and a light, which are attached to the bearing assembly.

The apparatus may include a wheel attached to the bearing assembly which can be manually rotated by the male partner. In this way the male partner can manually rotate the bearing assembly.

The apparatus may further include two handles attached to the bearing assembly for gripping by the hands of the female partner. The apparatus may further include two foot rests attached to the bearing assembly. The foot rests are designed and positioned to support the legs of the female partner.

There may also be a cover attached to the end of the bearing assembly, which covers at least a portion of the horizontal support.

The apparatus may further include a brake assembly positioned to slow and stop rotation of the bearing assembly. The brake lever is positioned for manual operation by the female.

The apparatus may further include a harness positioned to secure the female partner to the horizontal support.

The apparatus may further include a lock to limit rotation of the bearing assembly and another lock to limit sliding of the bearing assembly in relation to the base.

Preferably the horizontal support is removable from the bearing assembly and the entire apparatus is foldable.

The apparatus may further have a fully motorized version. The movements of the device, including rotational and back and forth sliding movement will be fully automated and motorized with the movement having a pre-set speed, direction or a combination of both. It can be remotely controlled using a wireless remote control or a wired remote control.

The apparatus may further have frame covers made of leather or similar fabrics, designed not only to enhance the apparatus aesthetically but also to provide a cozy feeling for the user. This frame cover can also have pouches to store the restraint system for the rider and also to store the accessories that may come with the device.

The apparatus may further have a vertical version wherein the rider is in a seating position as opposed to lying in a supine position. The driver or male partner will, in this version, be the one who will be in a supine position. This version allows usage of said device over a bed or any support surface that will support the male partner in a supine position. In this version the ring mechanism will be confined on the top portion and the female rider will now be supported mostly on the lower part of her body, from the legs to the buttocks. This version will have two types of stands to support the device. One type will have the device supported by two stands placed on each side of the rotating device. The other type will have just one stand with an overhang to which the rotating device will be attached to.

An appreciation of the other aims and objectives of the present invention and an understanding of it may be achieved by referring to the accompanying drawings and description of a preferred embodiment.

### BRIEF DESCRIPTION OF THE DRAWINGS

FIG. 1 is a perspective view of this invention.

FIG. 2 is a side elevational view of this invention showing how it is used.

FIG. 3 is a front elevational view of this invention showing how the female partner fits in the invention.

FIG. 4 is a front elevational view of this invention illustrating its clockwise rotation.

FIG. 5 is a front elevational view of this invention illustrating its counter-clockwise rotation.

FIG. 6 is a front elevational view showing how the rings can open for ingress and egress of the female partner.

FIG. 7 is a side elevational view showing how the bed can move backwards and forwards on the base.

FIG. 8 is a side elevational view showing attachment of accessories.

FIG. 9 is a perspective view illustrating mechanization of the invention.

FIG. 10 is a perspective view illustrating the optional cover for the invention.

FIG. 11 is a detail showing construction of the preferred rings of this invention.

FIG. 12 is a detail showing attachment of a hand brake.

FIG. 13 is a detail showing attachment of the brake caliper.

FIG. 14 is a perspective view showing addition of a height adjustment knob to this invention.

FIG. 15 is a side view of the height adjustment mechanism at its lowest point.

FIG. 16 is a side view of the height adjustment mechanism at a point above its lowest adjustment.

FIG. 17 illustrates a collapsible embodiment of this invention.

FIG. 18 shows the collapsible embodiment, partially collapsed.

FIG. 19 show a sectional view of an alternate ring construction for this invention.

### DETAILED DESCRIPTION OF THE PREFERRED EMBODIMENT

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

To completely understand the device and how it works, we must always keep in mind its main function: that is to assist the couple in achieving a certain position of intercourse. That position is the ability of the female organ to rotate continuously and to reciprocate, once the male organ has penetrated the female organ. The design of the device allows the female partner to lie down on her back and then support her legs while they are crossed, preferably just above the ankle. The female partner will be secured by a seat belt or any type of restraint including a corset design which enhances further the female rider's looks while supporting her whole body. Her legs may also be supported by a cover in the shape of a capsule or a leg rest in the form of a pouch or ankle support to maintain the cross-legs posture during usage and her arms will be supported by a pair of handle bars or a continuous bar designed for gripping by the rider. Once the female partner is secured inside the device, the male partner, after penetration, will now be able to rotate the female partner in either direction, either in a continuous circling motion, or just sideways from left to right or vice-versa. The device will also allow the rotating female partner to move back and forth to achieve a pumping while twirling movement.

Consequently, this device will now allow various positions to be performed including various combinations. One example is where the male partner positions the female partner upside down and thus enters her from behind. In addition, with the aid of this invention, the male partner now can rotate the female partner from left to right while moving her back and forth at the same time. The male partner can now also penetrate and pump the female partner while she is on her side.

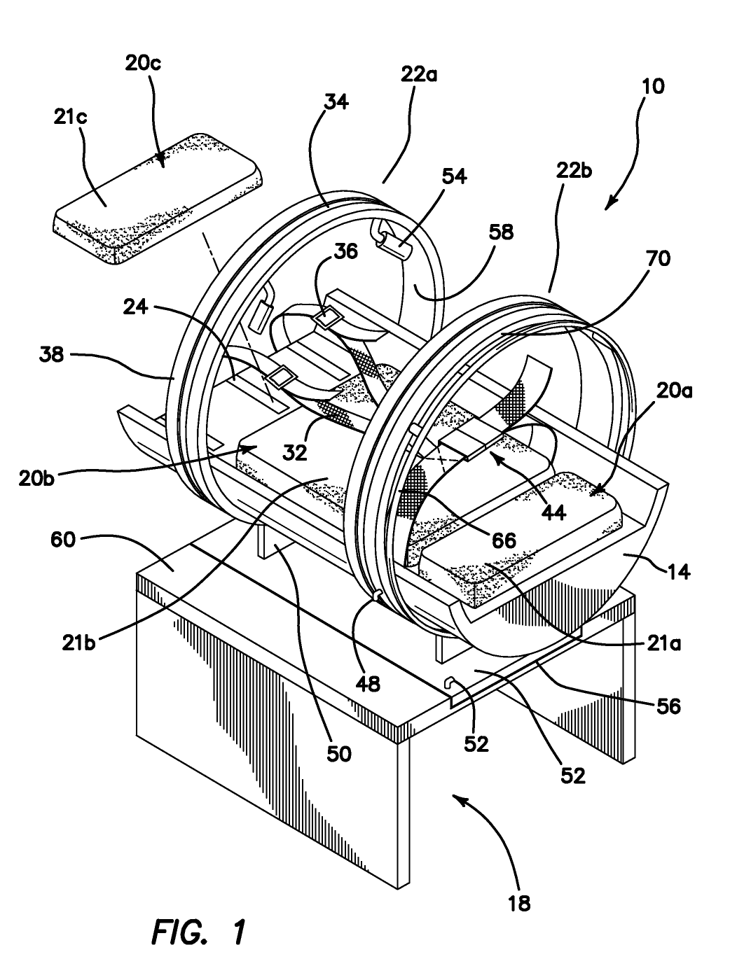

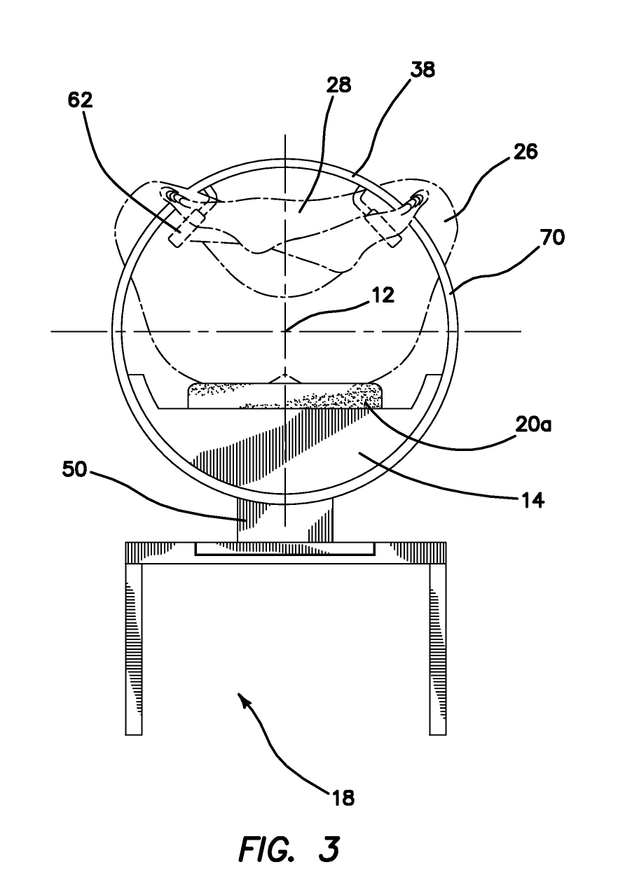

Referring to FIGS. 1, 2 and 3, this invention 10 includes a semi-cylinder 14 and a base 18, which is preferably collapsible.

One or more cushions 20a, 20b, 20c are positioned inside the semi-cylinder 14. Preferably the cushions are molded into a shape matching the female's body shape while in a semi-fetal position, with the legs 26 partly crossed, preferably about one to two inches just above the ankle 28. This position is the most convenient and most effective position for deepest penetration while keeping the female's legs 26 from hitting the male partner 30.

The cushion that supports the head 20c, may be detachable by means of, for example, hook and loop fastener 24. An adjustable harness 32 may be provided to keep the female 16 secured in place. This is necessary because of the rotational capability of the invention 10. The harness 32 will preferably go over the female's shoulders and waist. It may be tightened with buckles 36 snap fasteners 44 or the like.

The semi-cylinder 14 is made of a rigid material with sufficient strength to support the weight of the female 16 as well as to withstand the rotating motion and the back and forth motion of the semi-cylinder 14 during intercourse. The rigid material can be any hard plastic, any metal sheet, a metal framed in hard rubber, fiberglass, wood, Kevlar®, or any combination of these materials. The tops 21a, 21b, 21c of the cushions 20a, 20b, 20c function as the horizontal support of this invention 10.

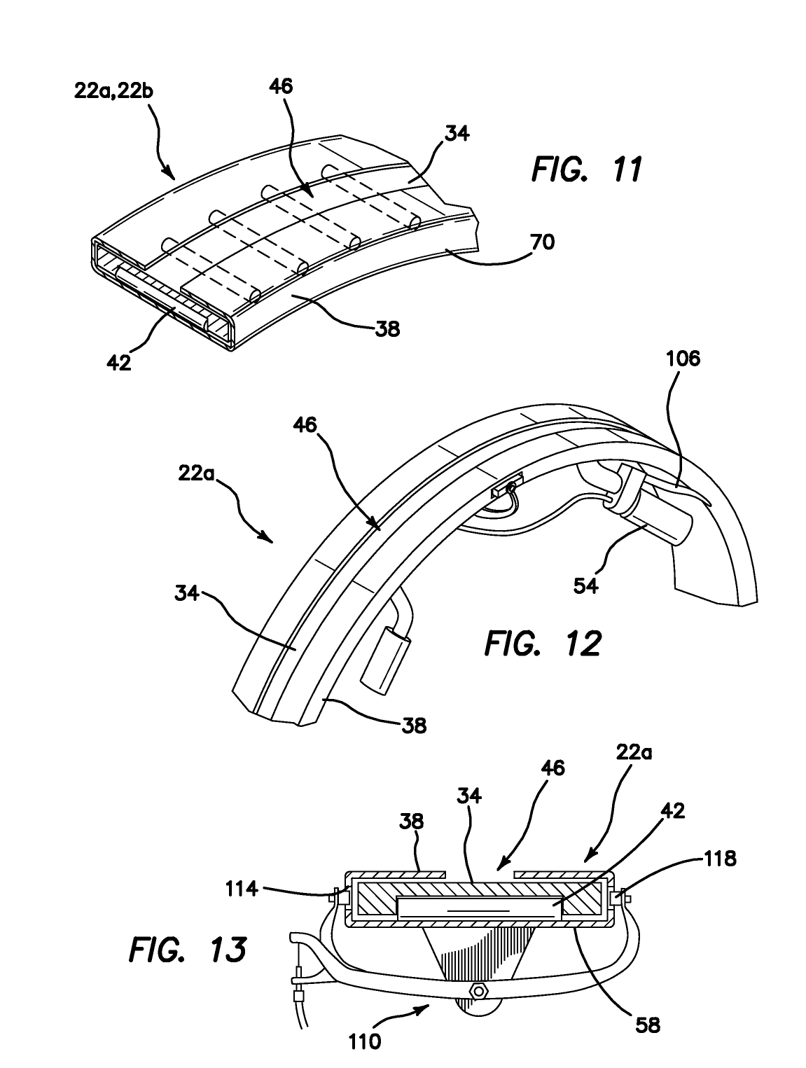

The semi-cylinder 14 is attached to at least a rear ring assembly 22a and a front ring assembly 22b which circle the semi-cylinder 14. These function as a bearing assembly for the invention 10. The preferred basic structure of the rings 22a, 22b is shown in FIGS. 11, 12 and 13. Each ring 22a, 22b comprises an inner ring 34, an outer ring 38 and a set of roller bearings 42 so they can rotate easily in relation to each other. The inner ring 34 is rectangular in cross section and the roller bearings 42 are set in it. The outer ring 38 has the cross section of a rectangular tube and wraps almost around the inner ring 34 leaving a slot 46. In this way the outer ring 38 protects the female partner 16 from touching moving parts and allows either partner 16, 30 to hold the ring 22a, 22b during operation.

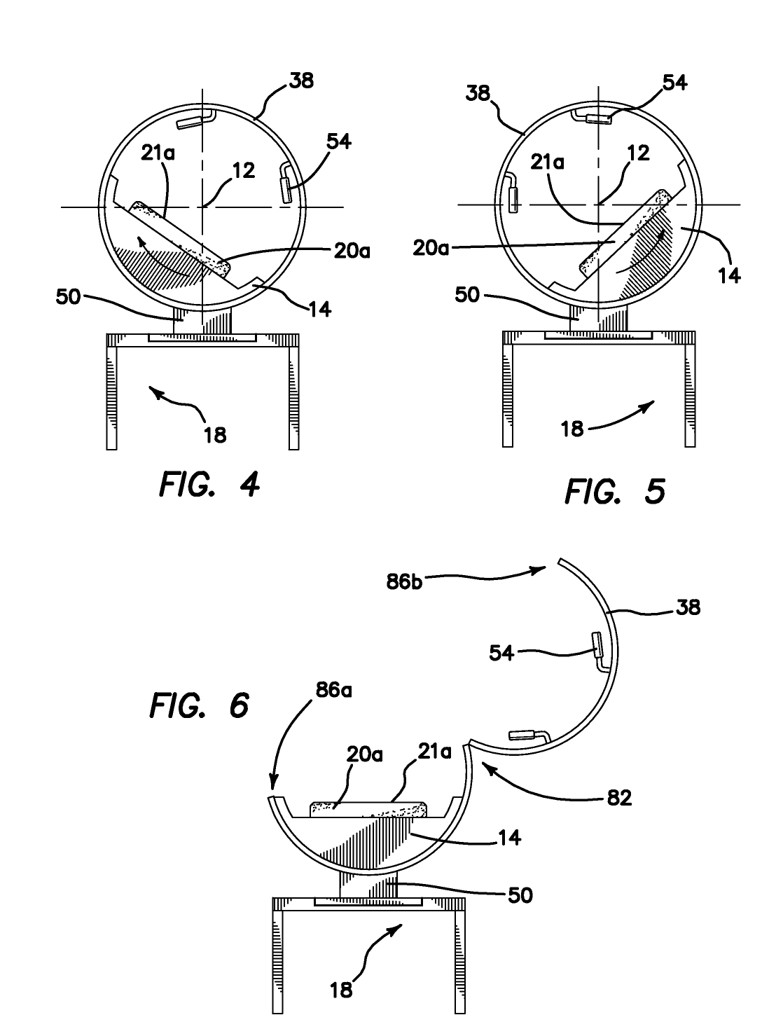

The inner ring 34 of each ring assembly 22a, 22b is attached to the base 18 with a vertical metal plate 50 through the slot 46 in the outer ring 38. Because of the construction of the ring assemblies 22a, 22b, the outer rings 38, the attached semi-cylinder 14 and cushion 20 can rotate as shown in FIGS. 4 and 5. A lock 48 may be provided to prevent or limit motion if desired.

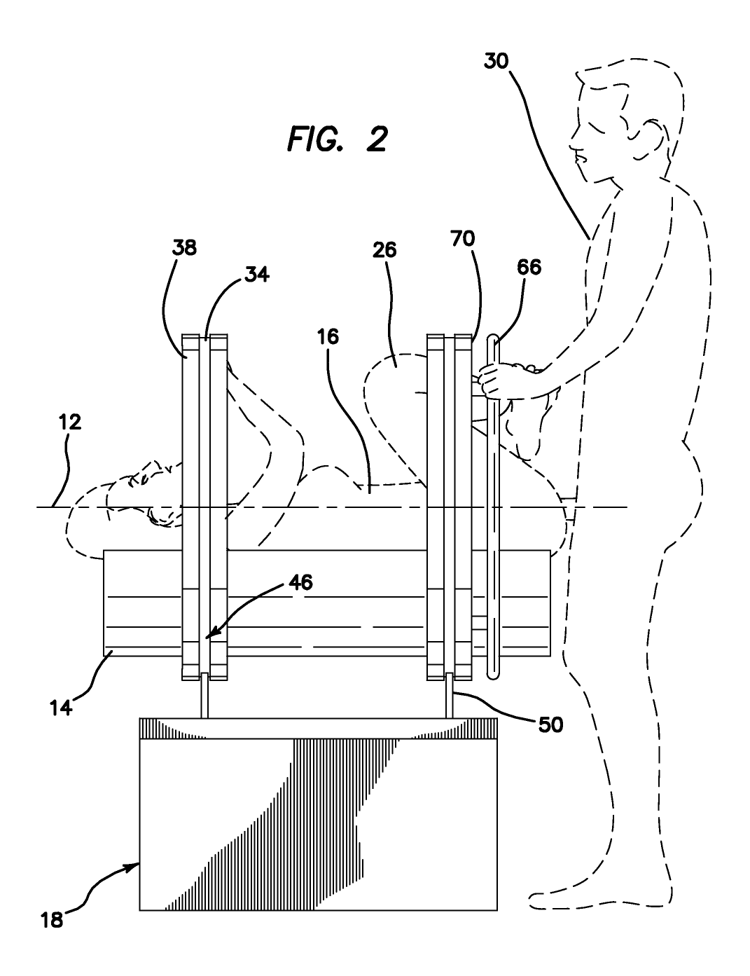

The tops of the cushions (horizontal support) 21a, 21b, 21c and rings (bearing assembly) 22a, 22b are designed and assembled so that the axis of rotation 12 is coaxial with the longitudinal axis of the sex organ of the female 16 when she is supported on the horizontal support in the supine position, as shown in FIGS. 2, 3, 4 and 5.

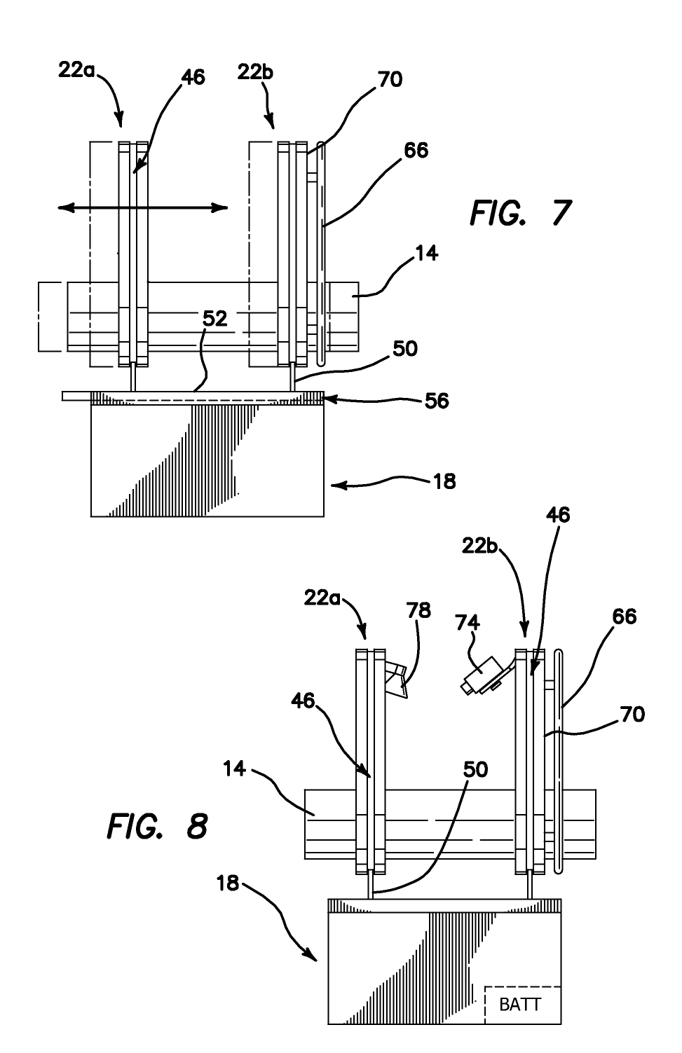

The plates 50 are attached to a slide 52 which fits into a channel 56 cut into the top 60 of the base 18. In this way the ring assemblies 22a, 22b, semi-cylinder, cushions 20a, 22b, 22c, and female 16 can slide backwards and forwards as shown by the arrow on FIG. 7. Preferably the back and forth motion is limited to between 2 to 10 inches. Also a lock 52 may be provided so that back and forth motion can be disabled.

There may be handles 54, attached to the inner surface 58 of the rear ring assembly 22a. The female partner 16 may grab these handles 54 during operation of the invention 10. There also may be foot rests 62 on the inner surface 58 of the front ring assembly 22b. The female partner 16 may rest her feet on these rests 62 during operation of the invention 10. The foot rests 62 mainly serve as an additional protection to avoid the female partner's legs 26 from accidentally hitting the male partner 30 and they also provide added comfort.

The ring assemblies 22a, 22b may be provided with hinges 82 and latches 86a, 86b so that the ring assemblies 22a, 22b can be opened for easy egress and ingress as shown on FIG. 6.

The invention 10 may include a wheel 66, attached to the front 70 of the front ring assembly 22b. The male partner 30 may manually turn the wheel 66 during operation of the invention 10. In this way the outer rings 38 the attached semi-cylinder 14, the cushion 20 and the female partner can be rotated as desired by the male partner 30.

The invention 10 may be accessorized with a variety of accessories such as a video camera 74 and a light 78 as shown on FIG. 8. Additionally, a front cover 90 may be provided for detachable attachment to the front of the invention 10.

The invention 10 may also be motorized. As shown in FIG. 9, to motorize it the front surface 70 of one of the rings 38 will be provided with gear teeth 94. Then an electric motor 98 with a matching gear 102 is mounted for co-operation with the teeth 94. Controls (not illustrated) will be mounted for operation by the male partner 30.

In addition there may be a brake for the invention. As shown in FIGS. 12 and 13 installation and operation will be similar to a caliper brake for bicycle. The brake handle 106 will be attached to one of the handles 54. The caliper 110 will be attached to the outer ring 38 and openings 114 will be provided through the outer ring 38 so that the pads 118 can apply braking force to the inner ring 34.

There will be a total of three motions available for this apparatus 10. These are a rotating motion, a back and forth motion and a twirling motion in which the semi-cylinder rotates as it is pushed and pulled by the male partner.

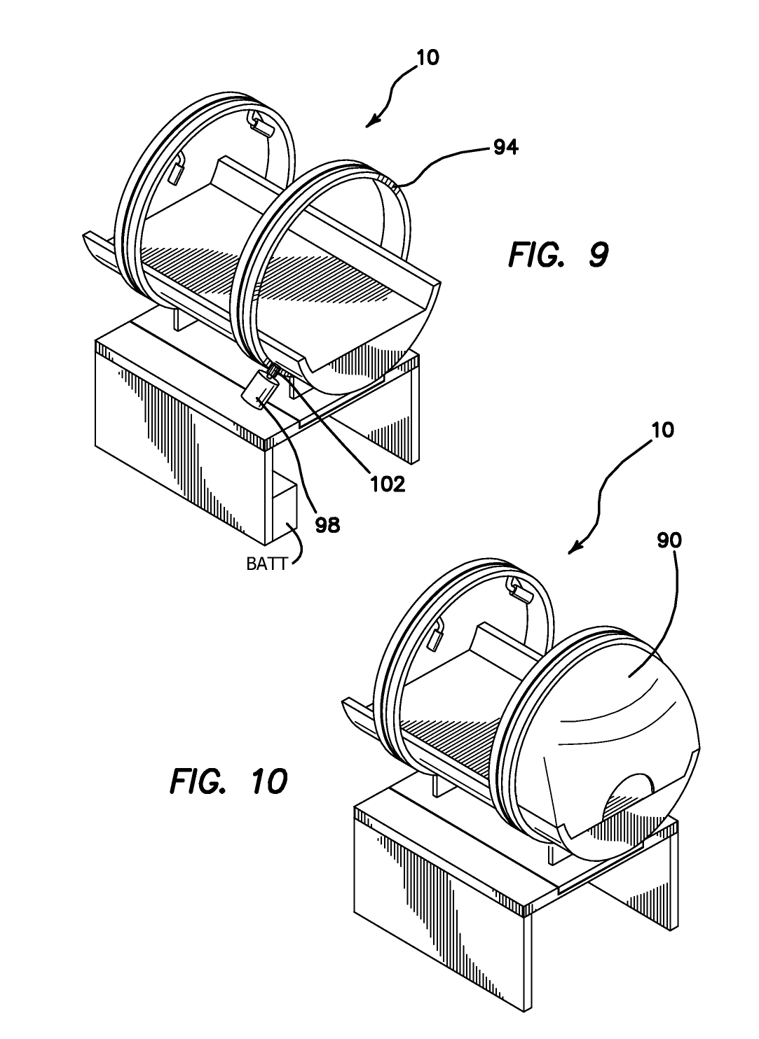

The main component, i.e. the semi-cylinder 14, and cushions 20a, 20b, 20c of this invention 10 can rotate through any desired angle. Preferably, during a 360 degree rotation, the legs 26 of the female 16 should be fully supported by a capsule-like encasement 90 as shown in FIG. 10. The purpose of this feature is for the comfort of the female and to avoid the accidental collision of the legs 26 with the male partner 30, especially when the female partner 16 is in an upside-down position.

The distance between the main component and the top 60 of the base 18 should be approximately 4 inches. The base 18 is preferably collapsible for portability. Preferably the height of the base should be adjustable so that the invention 10 can accommodate a male 30 of any height.

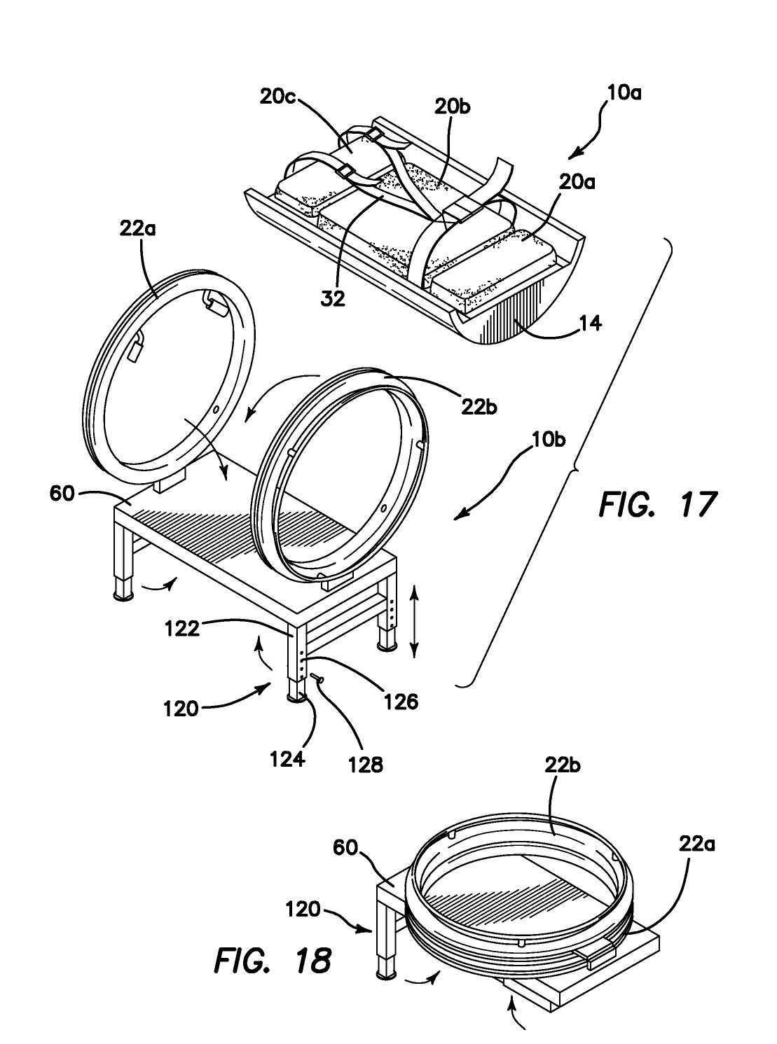

Preferably, the height of the invention is adjustable. Coarse height adjustment may be provided by adjustable legs 120, as illustrated on FIG. 17. A simple expedient is to provide inner 124 and outer 122 telescoping portions with a series of through holes 126. The positions of the portions 122, 124 are fixed by sliding a pin 128 through a set of aligned holes 126. Other height adjustment mechanisms, including screws, and motorized mechanisms are conceivable and could be employed.

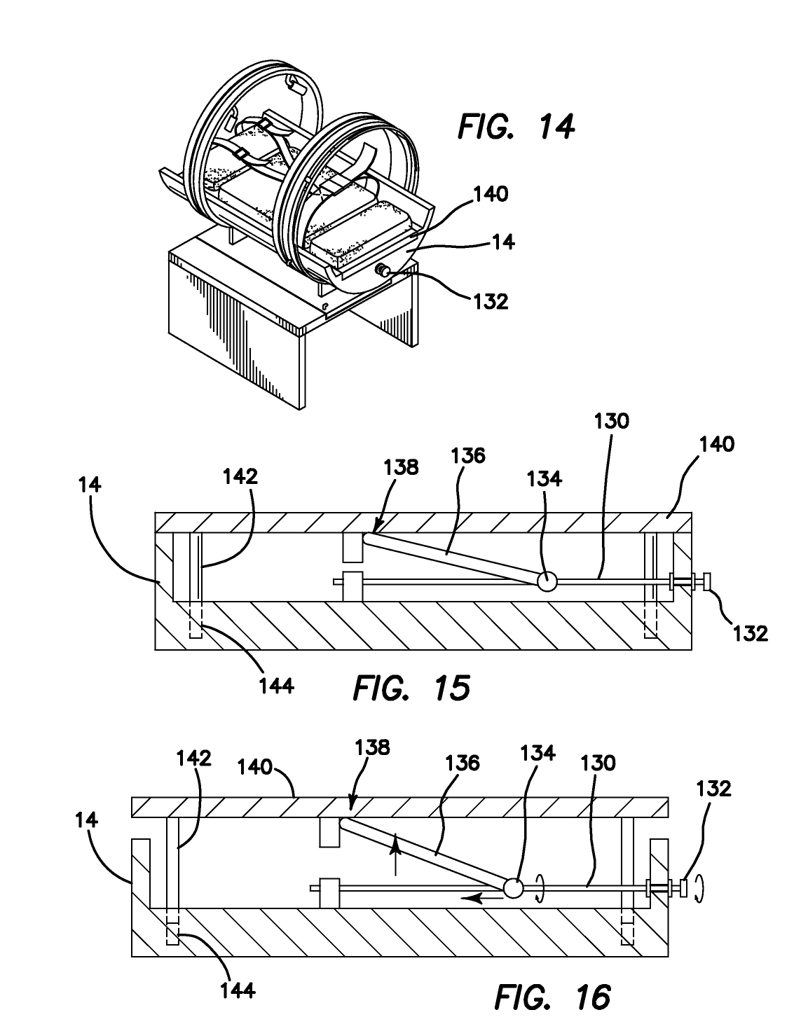

Fine height adjustment may be provided by a scissors mechanism as shown in FIGS. 14, 15 and 16. A threaded rod 130 is supported horizontally and connected to a knob 132. There is a threaded traveler 132 on the threaded rod, 130 and a lifting member 136 is pivotally connected between the traveler 132 and a fixed point on the top surface 140. As the threaded rod 130 is turned in the proper direction by the knob 132, the threaded traveler 134 moves inwards as shown by the arrow on FIG. 16, the lifting member 136 moves upwards, as shown by the arrow on FIG. 16 and the top surface 140 raises up. Turning the knob 132 in the opposite direction will reverse everything and the top surface 140 will lower. Sway bars 142 sliding in matching bores 144 may be necessary to keep the top surface 140 level and even as it raises and lowers. Other height adjustment mechanisms, including vertical screws and motorized mechanisms are conceivable and could be employed.

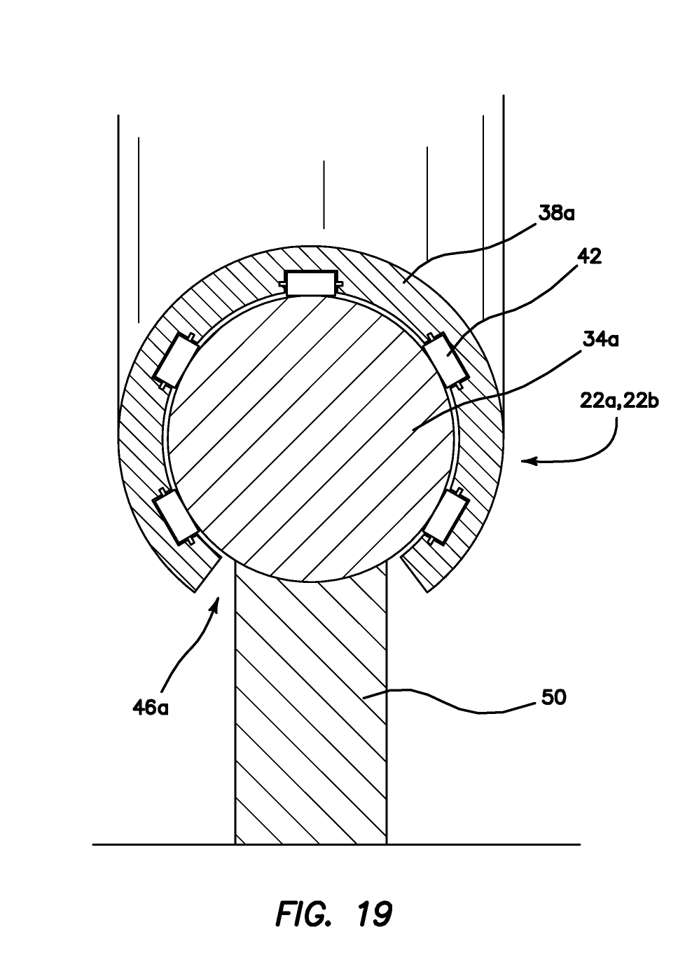

FIGS. 17 and 18 illustrate how the invention 10 is collapsible. The invention 10 is preferably separable into two subassemblies 10a, 10b. The bed subassembly 10a includes the semi-cylinder 14, cushions 20a, 20b, 20c and harness 32. The base subassembly 10b includes the rings 22a, 22b, which are foldable, and base 18. The base 18 comprises the top 60 and legs 120, which are foldable. After the bed subassembly 10a is removed from the base subassembly 10b, the rings 22a, 22b are folded in the directions shown by the arrows on FIG. 17. Then the legs 120 are folded in the directions shown by the arrows on FIG. 18. Those familiar with the art to which this invention pertains will understand that many alternative means for folding this invention 10 are conceivable and could be utilized.

FIG. 19 illustrates an alternate basic structure of the rings 22a, 22b. Each ring 22a, 22b comprises an inner ring 34a, an outer ring 38a and a set of roller bearings 42. The inner ring 34a has a circular cross section. The outer ring 38a has the cross section of a circular tube and wraps almost around the inner ring 34a leaving a slot 46. The roller bearings 42 are set in the outer ring 38a. The inner ring 34a of each ring assembly 22a, 22b is attached to the base 18 with a vertical metal plate 50 through the slot 46a in the outer ring 38a.

The following reference numerals are used on FIGS. 1 through 19:
10 rotating sex machine of this invention
10a bed subassembly
10b base subassembly
12 axis of rotation
14 semi-cylinder
16 female partner
18 base
20a front cushion
20b middle cushion
20c rear or head cushion
22 ring assembly
22a rear ring assembly
22b front ring assembly
24 hook and loop fastener
26 female's legs
28 female's ankles
30 male partner
32 harness for female partner
34 rectangular inner ring
34a circular inner ring
36 harness buckle
38 rectangular outer ring
38a circular outer ring
42 roller bearing
44 adjustable snap closure
46 slot in outer ring
48 ring lock
50 vertical metal plate
52 lock
54 handle
58 inner surface of ring assembly
60 top of base
62 foot rest
66 wheel
70 front of front ring assembly
74 video camera
78 light
82 ring hinge
86 ring latch
90 front cover
94 gear teeth
98 electric motor
102 gear
106 brake handle
110 caliper
114 brake pad openings in outer ring
118 brake pads
120 adjustable legs
122 outer leg
124 inner leg
126 through hole
130 threaded rod
132 adjustment knob
134 threaded traveler
136 lifting member
138 fixed point
140 top, moveable surface

Thus, the present invention 10 has been described herein with reference to particular embodiments for particular applications. Those having ordinary skill in the art and access to the present teachings will recognize additional modifications, applications and embodiments within the scope thereof.

It is therefore intended by the appended claims to cover any and all such applications, modifications and embodiments within the scope of the present invention.

What is claimed is:

1. An apparatus for use in performing intercourse comprising:
a) a horizontal support; said horizontal support designed to support a female in a supine position; a height of said horizontal support being adjustable;
b) a bearing assembly attached to an outside of said horizontal support; said bearing assembly designed to permit rotation of said horizontal support about an axis of rotation of said bearing assembly; said horizontal support and bearing assembly designed so that said axis of rotation is coaxial with a longitudinal axis of the sex organ of said female when she is supported on said horizontal support in said supine position; and
c) a base horizontally slidably attached to a bottom of said bearing assembly; a height of said base being adjustable;
whereby the height of said female's sex organ can be adjusted for penetration by a male partner's sex organ when said male partner is standing at an end of said horizontal support.

2. An apparatus as claimed in claim 1 in which said horizontal support includes a cushion.

3. An apparatus as claimed in claim 1 in which said bearing assembly is also openable.

4. An apparatus as claimed in claim 1 further comprising a video camera attached to said bearing assembly.

5. An apparatus as claimed in claim 1 further comprising a light attached to said bearing assembly.

6. An apparatus as claimed in claim 1 further comprising a wheel attached to said bearing assembly; said wheel adapted for manual rotation by said male partner; whereby said male partner can manually rotate said bearing assembly.

7. An apparatus as claimed in claim 1 further comprising at least one handle attached to said bearing assembly; said at least one handles adapted for gripping by the hands of said female partner.

8. An apparatus as claimed in claim 1 further comprising two foot rests attached to said bearing assembly; said foot rests adapted to support the legs of said female partner.

9. An apparatus as claimed in claim 1 further comprising a cover attached to said end of said bearing assembly; said cover covering at least a portion of said horizontal support.

10. An apparatus as claimed in claim 1 further comprising a brake assembly adapted to slow and stop rotation of said bearing assembly; said brake assembly including a brake lever; said brake lever adapted for manual operation by said female.

11. An apparatus as claimed in claim 1 further comprising a harness adapted to secure said female partner to said horizontal support.

12. An apparatus as claimed in claim 1 further comprising a lock to limit rotation of said bearing assembly.

13. An apparatus as claimed in claim 1 further comprising a lock to limit sliding of said bearing assembly in relation to said base.

14. An apparatus as claimed in claim 1 in which said horizontal support is removable from said bearing assembly.

15. An apparatus as claimed in claim 14, in which said apparatus is foldable.

16. A method of performing intercourse comprising the steps of:
a) providing a horizontal support; said horizontal support designed to support a female in a supine position; a height of said horizontal support being adjustable;
b) providing a bearing assembly; said bearing assembly designed to permit rotation of said horizontal support about an axis of rotation of said bearing assembly;
c) attaching said bearing assembly to an outside of said horizontal support; said horizontal support and bearing assembly designed so that said axis of rotation is coaxial with a longitudinal axis of a sex organ of said female when she is supported on said horizontal support in said supine position;
d) providing a base; the height of said base being adjustable;
e) slidably attaching said base to a bottom of said bearing assembly;
whereby the height of said female's sex organ can be adjusted for penetration by a male partner's sex organ when said male partner is standing at an end of said horizontal support.

17. A method as claimed in claim 16 further comprising the steps of providing a cushion and attaching said cushion to said horizontal support.

18. A method as claimed in claim 16 in which said bearing assembly is also openable.

19. A method as claimed in claim 16 further comprising the steps of providing a video camera and attaching said video camera to said bearing assembly.

20. A method as claimed in claim 16 further comprising the steps of providing a light and attaching said light to said bearing assembly.

21. A method as claimed in claim 16 further comprising the steps of providing a wheel adapted for manual rotation by said male partner and attaching said wheel to said bearing assembly; whereby said male partner can manually rotate said bearing assembly.

22. A method as claimed in claim 16 further comprising the steps of providing at least one handle adapted for gripping by the hands of said female partner, and attaching said handle to said bearing assembly.

23. A method as claimed in claim 16 further comprising the steps of providing two foot rests adapted to support the legs of said female partner and attaching said foot rests to said bearing assembly.

24. A method as claimed in claim 16 further comprising the steps of providing a cover adapted to cover at least a portion of said horizontal support, and attaching said cover to said end of said bearing assembly.

25. A method as claimed in claim 16 further comprising the steps of:
a) providing a brake assembly adapted to slow and stop rotation of said bearing assembly; said brake assembly including a brake lever; said brake lever adapted for manual operation by said female; and
b) attaching said brake assembly to said bearing assembly.

26. A method as claimed in claim 16 further comprising the steps of providing a harness adapted to secure said female partner to said horizontal support, and attaching said harness to said horizontal support.

27. A method as claimed in claim 16 further comprising the steps of providing a lock to limit rotation of said bearing assembly, and attaching said lock to said bearing assembly.

28. A method as claimed in claim 16 further comprising the steps of providing a lock to limit sliding of said bearing assembly in relation to said base; and attaching said lock to said base.

29. A method as claimed in claim 16 in which said bearing assembly is removably attached to said horizontal support.

30. A method as claimed in claim 16, in which said bearing assembly can additionally fold onto said base.
